(19) United States
(12) Patent Application Publication
(10) Pub. No.: US 2024/0123251 A1
(43) Pub. Date: Apr. 18, 2024

(54) TRANSCRANIAL MAGNETIC STIMULATION SYSTEM AND METHOD

(71) Applicant: Ampa Inc., San Diego, CA (US)

(72) Inventors: Donald A. VAUGHN, San Diego, CA (US); Jonathan A.S. DOWNAR, Carrying Place (CA)

(21) Appl. No.: 18/212,081

(22) Filed: Jun. 20, 2023

Related U.S. Application Data
(63) Continuation-in-part of application No. 17/964,686, filed on Oct. 12, 2022, now Pat. No. 11,730,969.

(51) Int. Cl. A61N 2/02

(52) U.S. Cl. CPC A61N 2/02

(57) ABSTRACT

Disclosed is a transcranial magnetic stimulation (TMS) coil head configured to be placed over a target brain region for treatment, wherein the TMS coil head includes a housing containing one or more coil windings within the housing, wherein said one or more coil windings are configured to generate a maximum magnetic field at a common focal point, and one or more imaging devices including a single camera configured to overlie the common focal point is as to permit direct visualization of a position of the TMS coil head relative to a target position on a patient's head.

### CROSS REFERENCE TO RELATED APPLICATION

This application is a Continuation-in-Part (CIP) of U.S. Application Ser. No. 17/964,686, filed Oct. 12, 2022, the contents of which are incorporated herein by reference.

### BACKGROUND OF THE DISCLOSURE

The present disclosure relates to a system for transcranial magnetic stimulation (TMS), i.e., methods and apparatus for positioning a transcranial magnetic stimulation device properly on the head of patients so as to deliver magnetic stimulation to a specific brain region. The disclosure has particular applicability to systems and methods for applying magnetic stimulation to target brain regions of a patient for treating depression and will be described in connection with such utility, although other utilities are FDA-approved including treatment for obsessive compulsive disorder (OCD), depression with comorbid anxiety, and nicotine addiction, and still others show promising early results including TMS for treatment of various neurobehavioral disorders including Major Depression Disorder (MDD), Obsessive-Compulsive Disorder (OCD), bipolar disorder, post-traumatic stress disorder (PTSD), eating disorders, personality disorders, alcohol, drug and other substance use disorders, gambling, smoking cessation, mild cognitive impairment, Asperger's Syndrome, Multiple Sclerosis (MS), ALS, Tourette's Syndrome, blepharospasm, stroke, and autism, Alzheimer's Disease and other dementias, migraine headaches, movement disorders such as Parkinson's Disease, tinnitus, chronic pain, ADHD, OCT and epilepsy.

For each of these disorders, a distinct set of brain regions is known to be functioning abnormally, and one or more of these regions must be located and accurately targeted during stimulation for successful treatment. In order to reliably stimulate a desired brain region, the TMS coil or coils need to be consistently and accurately placed at a target scalp location overlying that brain region, i.e., so that the maximum field strength of the coil or coils falls on the target brain region, and simultaneously induce an electrical field intensity in the relevant brain tissue which is high enough to induce neuronal stimulation, without adversely effecting other regions of the brain, and must remain at that site throughout the entire stimulation session. Stimulation of off-target brain regions may reduce or eliminate the efficacy of the treatment, and in the worst case scenario may lead to worsening of symptoms, excessive pain during treatment, or rarely, a serious adverse event such as a seizure.

The process of positioning the TMS coil on the head, and maintaining it in place during treatment, is known as ‘neuronavigation.’ In current clinical practice, the most common approach to neuronavigation is to place a fabric cap on a patient's head, perform measurements of the head and scalp, and use these measurements to define a coordinate system (e.g., the ‘10-20 international encephalography system’) which provides markers on the patient's head. A TMS operator would then use these marks to specify the target site on the cap, place the TMS coil over the marked target site, and then trace a (partial) outline of the coil on the cap for use in maintaining consistent coil orientation during treatment sessions, and from session to session. However, this method may be imprecise, and does not provide direct visual confirmation that the center of the coil is (1) directly over the target location, or (2) actually in physical contact with (i.e., touching) the patient's head.

In addition, patients may move during treatment and/or navigational aids may slip out of the desired position. If this occurs, the TMS technician must pause treatment, readjust the position of the coil, and then resume stimulation. At worst, a patient can move his or her head in such a way that the TMS coil moves but is not observably positioned off target such that the session continues, with potential adverse consequences as identified above. Present methods lack a direct visual or other record confirming that (1) the TMS coil is optimally located over the desired target area, (2) the TMS coil remains over the desired target area throughout the stimulation session, and that (3) the TMS coil remains in physical contact with the surface of the patient's head during the entire session.

A more complex, less commonly used approach to neuronavigation (FIG. 1) involves a computerized frameless stereotaxic positioning system comprised of: (1) a set of optical position markers such as small reflective beads 70 attached in a specific 3-dimensional configuration to the coil as well as to a tracker on the patient's head, (2) a stereo camera 71 that visualizes and localizes the markers in 3-dimensional space, and (3) computer software 72 which uses 3-dimensional marker position information from the camera to impute the relative positions and orientations of the patient's head and the coil, and then provides a visualization of these positions on a screen viewed by the operator as a neuronavigation guide before and during treatment. Such a system usually, but not always, also includes (4) an MRI or other 3-dimensional image of the patient's head and brain, which the software aligns with the 3-dimensional imputed head position, so that the operator may visualize the brain region at the focus of the coil in real-time during coil positioning and treatment.

As examples, JP 2003-180649A and JP 2004-000636A disclose techniques for TMS coil neuronavigation using, for example, an optical tracking system employing infrared reflectors, as described above. This technology is commercially available and is also used in clinical settings such as neurosurgical procedures requiring neuronavigation.

JP 2006-320425A discloses another apparatus for positioning a TMS coil against the patient's head by using a multi jointed robot. This approach likewise has several major disadvantages, including the necessity of an MRI scan for every patient, the excessive additional expense and complexity of the apparatus itself, and the need for an operator to undergo an extended training period of several additional weeks to achieve proficiency in accurate use of the system. Further, the system can fail if: (1) the specified target is mistaken, (2) the markers on the coil are incorrectly calibrated, (3) following calibration, the markers on the patient's head move out of position during the session, (4) the operator is insufficiently skilled, or if (5) the coil is not quite in contact with the scalp despite appearing to be so on the neuronavigation system.

The complexity of this external tracking approach with MRI-guidance also greatly reduces the variety, and hence numerosity, of locations where patients may receive TMS treatment. This system is typically confined to a hospital setting because it requires high-field MRIs, as well as significant computing resources, specialized analysts to process the images, and technicians trained to competently operate the cumbersome neuronavigation suite. As a result, this approach is rarely used in the most accessible health care settings such as primary care clinics, mental health centers, assisted living facilities, outpatient specialty clinics, or workplace health centers. Instead, a patient seeking MRI-guided neuronavigated TMS is generally obliged to repeatedly travel to an academic or tertiary health care setting, raising additional barriers of cost and convenience and curtailing the accessibility of TMS treatment for those who need it.

The foregoing discussion of the prior art derives in part from U.S. Pat. No. 10,004,915 (the '915 Patent) wherein there is described a TMS system comprising a TMS alignment system comprising a means for generating magnetic field, the magnetic field generating means having a coil for generating a variable magnetic field to be applied to a certain part of patient's head and a holder for holding the coil; and a camera means for recognizing a predetermined reference marking made on a specific portion of the ear of the patient, (e.g., the tragus); the magnetic field generating means and the recognizing means being designed so that an alignment of the recognizing means with the marking causes the coil to be set in a proper posture with respect to the certain part of the patient's head.

According to the '915 Patent, with the aforesaid arrangement, the magnetic field generating means can be positioned with respect to the reference marking of a specific portion the patient's ear, allowing the user of the TMS system to position the magnetic field generating means without skill which is needed for conventional systems.

The recognition means of the '915 Patent includes at least one imaging device, i.e., cameras carried on external arms extending from an apparatus. Alignment includes aligning an optical axis of the imaging device with the marking. This allows the coil to be positioned in the proper posture with respect to the specific part of the patient.

Preferably, the TMS system of the '915 Patent further comprises an optical device capable of emitting a directional beam, the optical device being provided adjacent the imaging device, wherein the alignment includes aligning an intersection of the optical axis of the optical device with the marking. This allows the TMS coil to be positioned in the proper position with respect to the specific part of the patient.

In another embodiment of the '915 Patent, the TMS system further comprises a moving mechanism for moving the coil holder on and along a surface of the patient's head; and a controlling means for controlling the moving mechanism in accordance with an output from the recognition means to automatically position the holder with respect against the marking.

As noted earlier, problems with the TMS alignment system proposed in the '915 Patent is that the system requires marking directly on the patient, the system is bulky, and the arms required for holding the cameras or imaging devices are bulky and themselves prone to bending and/or misalignment. The alignment markings on the patient also can be obscured by the patient's hair. The additional components also bear the risk of inaccurately imputing the coil's actual position, as explained above. Lastly, the additional components reduce the overall accessibility of TMS treatment, by requiring technicians to undergo extensive additional training in order to operate the neuronavigation apparatus correctly, and by limiting treatment location to areas where a stationary multi-ton MRI scanner happens to reside.

In addition to coil placement, the art also has developed specifically designed coils for targeting specific brain regions including deep neuronal structures with minimal effect on other brain regions. Examples of specific brain regions that may be stimulated including frontal lobe regions, occipital lobe regions, parietal lobe regions, right temporal regions and left temporal regions in a certain circumference of the brain, such as around a particular axial slice. See for example, US Patent Publications Nos. 2014/025926; US 2014/025928; US 2014/025928; US 2016/0206895; and US 2016/0206896 which are given as exemplary. Of course, such specifically described coils still must be properly placed for maximum effect.

### SUMMARY OF THE INVENTION

In accordance with the present disclosure we provide a system for overcoming problems of the prior art by providing a TMS coil with an imaging device, i.e., a camera, configured to permit direct visualization of the position of a coil relative to a position on the patient's head so as to locate the maximum field strength of the coil, i.e., the focal point of the coil, directly over the target region of the brain.

More particularly, in accordance with Aspect A of our disclosure, we provide a TMS coil head configured to be placed over a target brain region for treatment, wherein the TMS coil head comprises a housing containing one or more coil windings within the housing, wherein said one or more coil windings are configured to generate a maximum magnetic field at a common focal point; and one or more imaging devices including a single camera configured to overlie the common focal point so as to permit direct visualization of a position of the TMS coil head relative to a target position on a patient's head.

In one embodiment the TMS coil head comprises two coil windings located to either side of the center of the TMS coil head. In such embodiment the two coil windings preferably are mirror images of one another.

In another embodiment the one or more imaging devices comprises one or more cameras, including the single camera overlying the common focal point of the one or more coil windings. In such embodiment the one or more imaging devices preferably comprises one or more visible light imaging cameras, one or more ultraviolet light imaging cameras or one or more infrared imaging cameras, and/or the one or more imaging devices also comprises two or more cameras located to sides of the TMS coil head. Alternatively, the one or more imaging devices may also comprise two or more cameras located away from the center but within the housing of the TMS coil head.

In another embodiment, the TMS coil head comprises one or more accelerometers configured to sense orientation placement or changes in orientation of the TMS coil head.

In still another embodiment, the TMS coil head comprises one or more contact sensors configured to detect contact and force between the TMS coil head and the patient's head. In such an embodiment, the contact sensors preferably comprise one or more force-sensitive resistors, one or more capacitive touch sensors or one or more ultrasonic position/touch sensors.

In yet another embodiment, the TMS coil head further comprises one or more imaging devices configured to permit simultaneous visualization of the patient's head as well as the TMS coil head. In such an embodiment, the one or more imaging devices preferably comprises one or more cameras, one or more LIDAR detectors, or one or more ultrasonic detectors.

In another embodiment the TMS coil head further comprises a memory device configured to create a record of the TMS coil head position before and during treatment.

In a preferred embodiment we also provide a contact sensor configured to determine whether the TMS coil remains in the same position and in contact with the scalp during the entire TMS stimulation session. In one embodiment, we provide an optical scalp-landmarking approach which allows for much higher consistency in positioning a TMS coil over a given site on the scalp from session to session, as well as providing a direct visual record (as opposed to an imputed calculation) of whether the coil was properly positioned and maintained in this position and in contact with the scalp consistently throughout each session of stimulation. That is to say, rather than using externally placed sensors and markers on the head to infer the coil location from an external perspective (as in FIG. 1), our approach uses sensors placed on the coil itself to, in essence, provide the coil's perspective. Moreover, since our novel approach allows the technician and supervising physician to directly visualize the target site while placing the coil, the time required to train a new technician to proficiency is greatly reduced, rather than extended. Moreover, a verifiable record of placement accuracy may be generated during each treatment session. Finally, our approach avoids the prior art use of costly and cumbersome additional components such as stereo cameras, MRI machines and intensive processing software, or reflector markers requiring calibration prior to treatment. This reduction in cost and complexity, as well as the marked acceleration of an operator's learning curve to proficiency, facilitates more widespread access to neuronavigated TMS treatment in a broader range of settings outside the more limited number of specialized centers which host large, expensive apparatus, requiring extensive personnel.

In another embodiment of the disclosure, in addition to providing an imaging device configured to provide direct visualization of a coil to locate the focal point of a coil at the target region of the patient's brain, we also incorporate two or more cameras placed off-center and/or on the sides of the TMS coil. The camera(s) may comprise visual light imaging capabilities, ultraviolet light imaging capabilities or infrared light imaging capabilities.

In accordance with another embodiment of our disclosure, we also incorporate into the TMS coil one or more contact sensors configured to detect whether the coil is in contact with the patient's head before, during, and until the treatment session concludes. The contact sensors may comprise force-sensitive resistors, capacitive touch sensors, ultrasonic position/touch sensors, and/or thermal/infrared sensors.

In another embodiment of our disclosure, we also incorporate one or more imaging devices external to the coil, configured to allow for simultaneous visualization of the patient's head (and any associated markings) as well as the coil, as an independent measure of their relative positions. These additional coil-external cameras may comprise one or more cameras, LIDAR detectors, and/or ultrasonic detectors.

In yet another embodiment of our disclosure, we provide a specialized treatment cap having indicia with various markings including grid markings, text and/or color markings corresponding to specific anatomical locations on the head of the patient.

In yet another embodiment of our disclosure, we provide a treatment cap geometry where the brim of the cap comes to a point on the midline. This point can immediately be visually confirmed to be correctly placed or not without the need for measuring tape. Additionally, we place indicia on the cap that denotes where the cap should be with respect to the tragus (the point flap of skin on the ear), as an additional marker to ensure reliable fit of the cap on the head.

With both the point front, and tragus markers on both sides, these three markers can be used not only for basic visual confirmation, but we can employ AI algorithm to ensure proper cap positioning, by using a smartphone camera and slowly wave it around the patient from left to front to right side to ensure the cap is properly positioned.

In yet another embodiment of our disclosure, the TMS system is configured to record and optionally, transmit, in real time, a video of the TMS coil placement during treatment. Also, in still yet another embodiment, we include one or more accelerometers in the TMS coil configured to provide a supplementary record of the orientation of the TMS coil throughout treatment, so that the provider can detect any subtle drift or deviation of the coil during treatment and make adjustments to the TMS coil orientation accordingly.

In another embodiment of our disclosure, we provide a transcranial magnetic stimulation system comprising: a TMS system configured to generate a magnetic field to be applied to a patient's brain region, the TMS system comprising a TMS pulse generator as well as an inductor coil; an imaging device incorporated into the coil and configured to permit direct visualization of the TMS coil to locate the coil on the patient's head so that the focal point of the coil is at the target region of the patient's brain. The imaging device may comprise a camera, preferably a visible light imaging camera, an ultraviolet light imaging camera, or an infrared imaging camera.

In another embodiment of our disclosure, the TMS system may further comprise one or more accelerometers configured to sense orientation placement and/or changes in orientation of the TMS coil.

In a further embodiment of our disclosure, the TMS system also may further comprise a memory device configured to create a video record of TMS coil placement during treatment.

We also provide a transcranial magnetic stimulation neuronavigation kit, comprising a TMS system as above described, and a patient head cap having grid markings, text and/or color markings configured to overlie anatomical locations on the head of the patient. The patient head cap may include markings configured to overlie target areas of the head of the patient, and/or markings configured to permit continuous measurement of the position and orientation of the cap relative to the patient's head before, during, and after treatment.

### BRIEF DESCRIPTION OF THE DRAWINGS

Further features of the disclosure will be seen from the following detailed description, taken in conjunction with the accompanying drawings, wherein:

FIG. 1 is a schematic view of a conventional frameless stereotaxic MRI-guided neuronavigation system, in accordance with the prior art;

FIG. 2 is a schematic view of a TMS system in accordance with an embodiment of our parent application;

FIG. 3 is a schematic view of a TMS coil in accordance with an embodiment of our parent application;

FIG. 4 is a bottom plan view and FIG. 4A a side elevational view of a TMS coil element of with an embodiment of our parent application;

FIG. 5 is a block diagram of a power and control circuit of our present disclosure;

FIGS. 6A and 6B are perspective views of a cap element of the present disclosure;

FIG. 7A-7D are perspective views of alternative cap elements of the present disclosure; and

FIGS. 8-16 are perspective views of various TMS coils in accordance with the present disclosure.

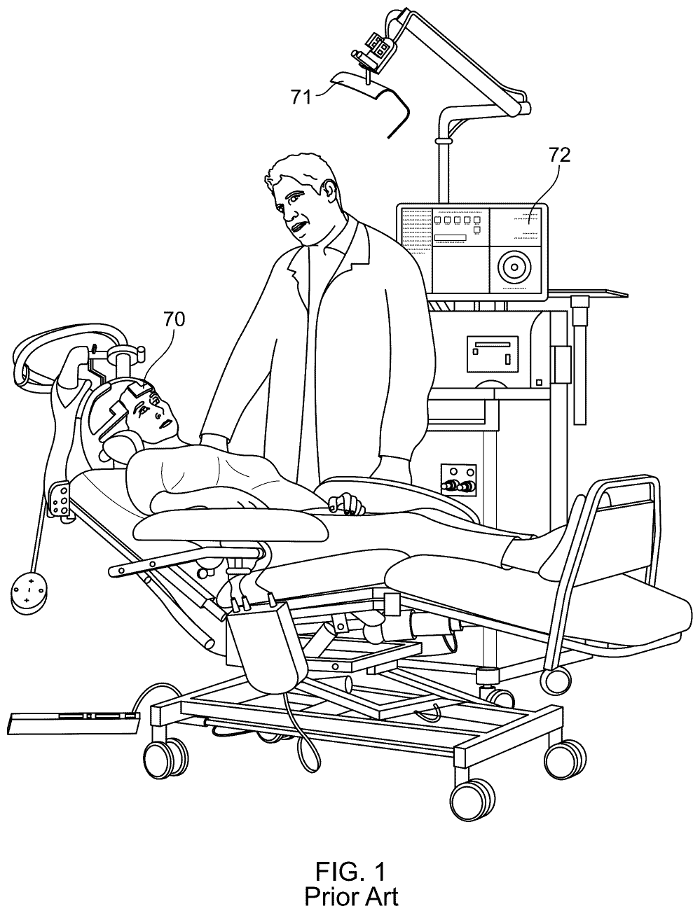

### DETAILED DESCRIPTION OF THE DISCLOSURE

Spatially relative terms, such as “inner,” “outer,” “beneath,” “below,” “lower,” “above,” “upper,” “base,”, “top”, and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly

As used herein the term except as otherwise stated transcranial magnetic stimulation (TMS) coil or coils and TMS coil head shall mean a single magnetic induction coil or plural coils and their housing(s).

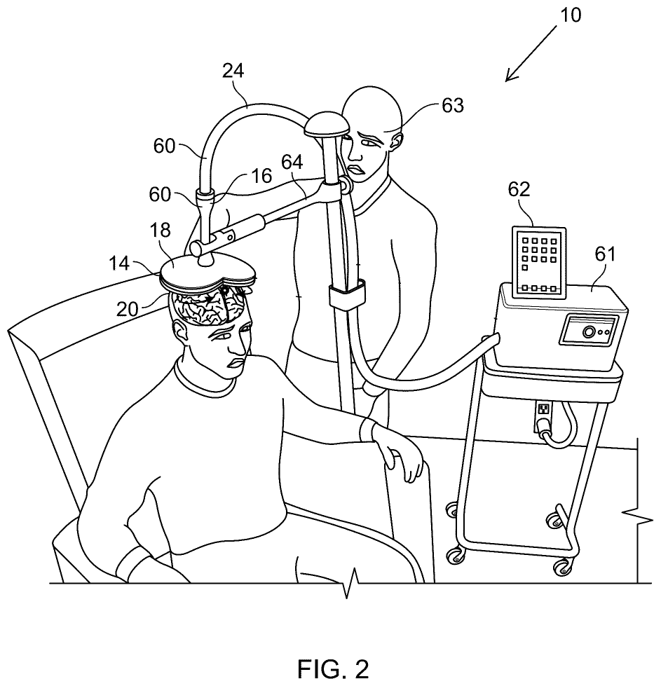

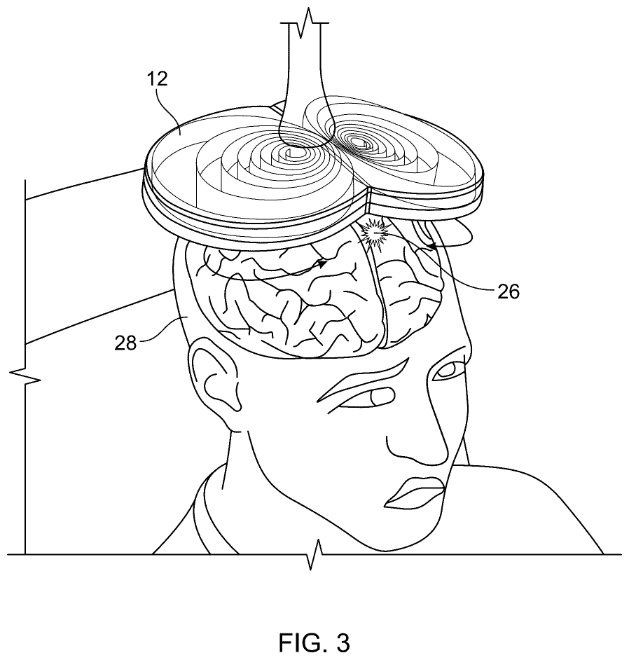

Referring to FIGS. 2-5, a neuronavigated transcranial magnetic stimulation system 10 of our parent application includes one or more TMS coils, which itself consists of two magnetic induction coil windings 12A, 12B in a housing 14. Alternatively, housing 14 may be connected to an inanimate support mechanism. Housing 14 includes a handle 16 sized for a human hand. Housing 14 includes a top surface 18 and a bottom surface 20, which can be arched to facilitate closer mating with the head of a patient.

The neuronavigated transcranial magnetic stimulation system 10 also includes a pulse generator 61 with an internal control unit and associated power source. The pulse generator sends electricity to windings 12 through a cable 24. The pulse generator 61 may be configured to communicate with smartphone, tablet or PC 62 having a program for the device to send parameters to the pulse generator 61 or for the device to receive data back from the pulse generator. The neuronavigated transcranial magnetic stimulation system 10 is designed to treat and/or ease certain symptoms by applying magnetic stimulation with a certain intensity and frequency through a patient's skull to a target area 26 in the brain within the patient's skull 28. The coil 60 may be held in place by an operator 63, coil-holder 64, or both.

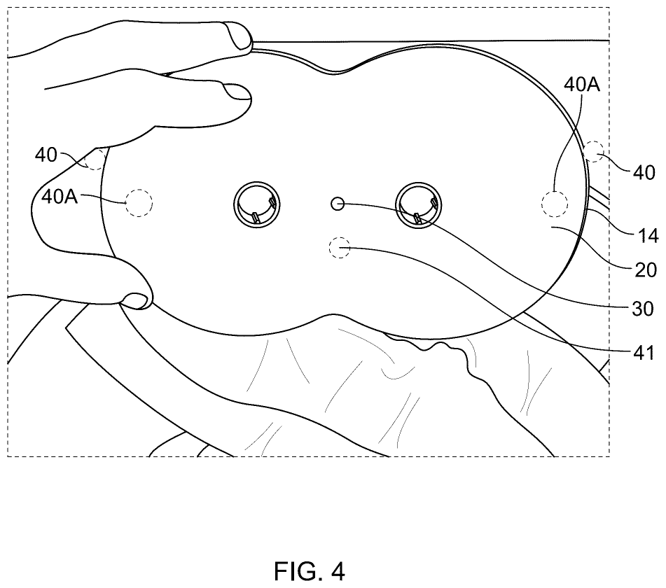

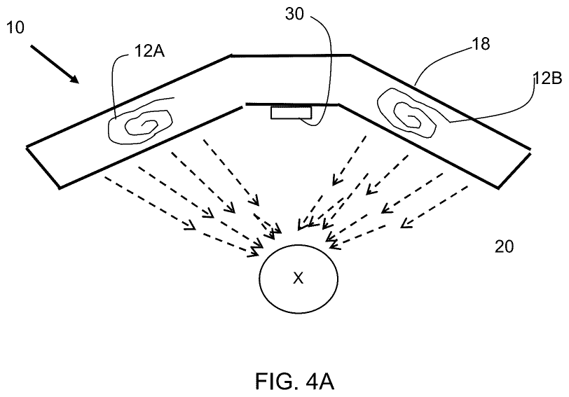

Referring in particular to FIG. 4, housing 14 includes an imaging device 30 configured to face downward from bottom surface 20, i.e., towards the head of a patient when in use, to permit direct visualization of the patient's head. In one embodiment, an imaging device 30 is located centrally relative to the two magnetic induction coil windings 12A, 12B (FIG. 4A). Imaging device 30 preferably comprises a camera which may be a visual light imaging camera, an ultraviolet light imaging camera or an infrared imaging camera.

Referring also to FIG. 4A, coil windings 12A, 12B direct energizing direct magnetic stimulation to a single focal point corresponding to target area “X”. As seen in FIG. 4A, imaging device, i.e., camera 30 is located directly over the focal point “X” target area “X”. This permits direct visualization of placement of the TMS coil head 10 so that the focal point of the coils, i.e., the maximum field strength of the coils lies on the target area “X” of the patient.

As shown in phantom at 40 (FIG. 4), the imaging devices also may include spaced imaging devices located away from the center, at the sides of the magnetic induction coil's windings 12A, 12B. Alternatively, two or more imaging devices shown in phantom at 40A, may be placed facing downward, away from the center of the housing, but within the housing spaced from one another at the same distance from the center of the windings 12A, 12B, or attached adjacent to the edges of the windings 12A, 12B.

Also, if desired, one or more contact sensors 41 (FIG. 4) configured to detect force between the coils and the patient's head may be provided, carried on the underside of housing 14. The contact sensors 41 may comprise one or more force-sensitive sensors, one or more capacitive sensors, or one or more infrared sensors.

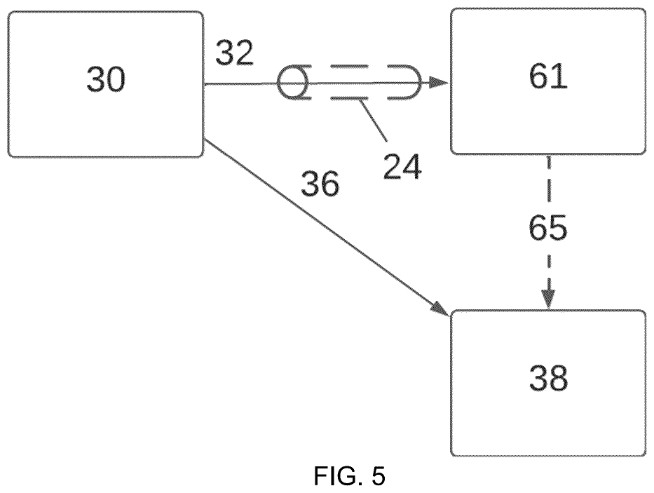

Referring in particular to FIG. 5, in one embodiment, imaging device 30 is connected via cable 36 to a display and memory device 38 which may be a smartphone, tablet or PC. In another embodiment, imaging device 30 is connected via a cable 32 to the pulse generator 61. Cable 32 may travel through cable 24. Optionally, pulse generator 61 may pass imaging information to device 62 which may be a smartphone, tablet or PC. This connection 65 may be wired, or wireless via Bluetooth, Wi-Fi, NFC or the like.

A feature and advantage of the present disclosure derives from use of one or more imaging devices internal to the TMS coil housing including an imaging device directly over the focal point of the coils not only ensures proper placement of the transcranial magnetic stimulation system, but also permits continuous monitoring of placement and includes an ability to record and/or transmit placement data in real time during the entire procedure.

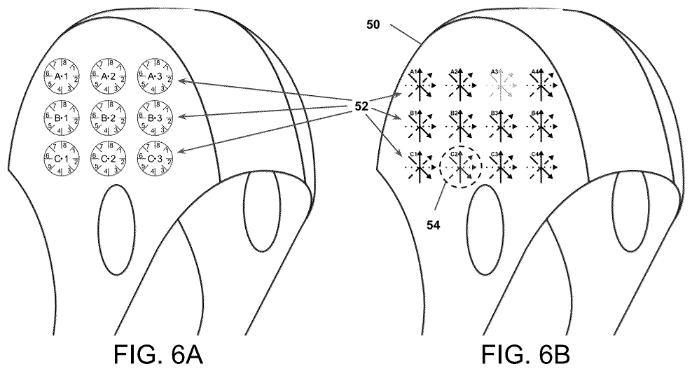

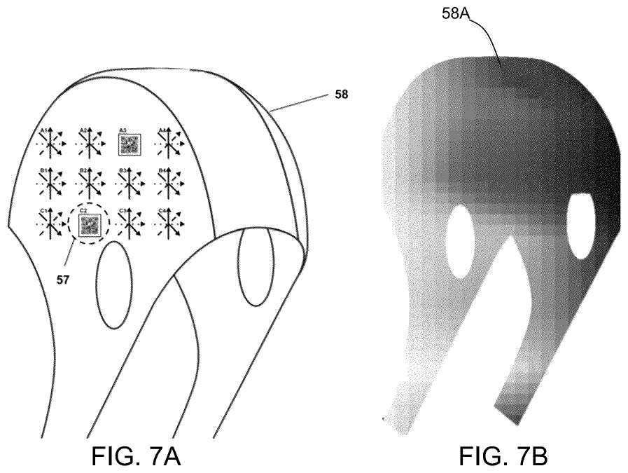

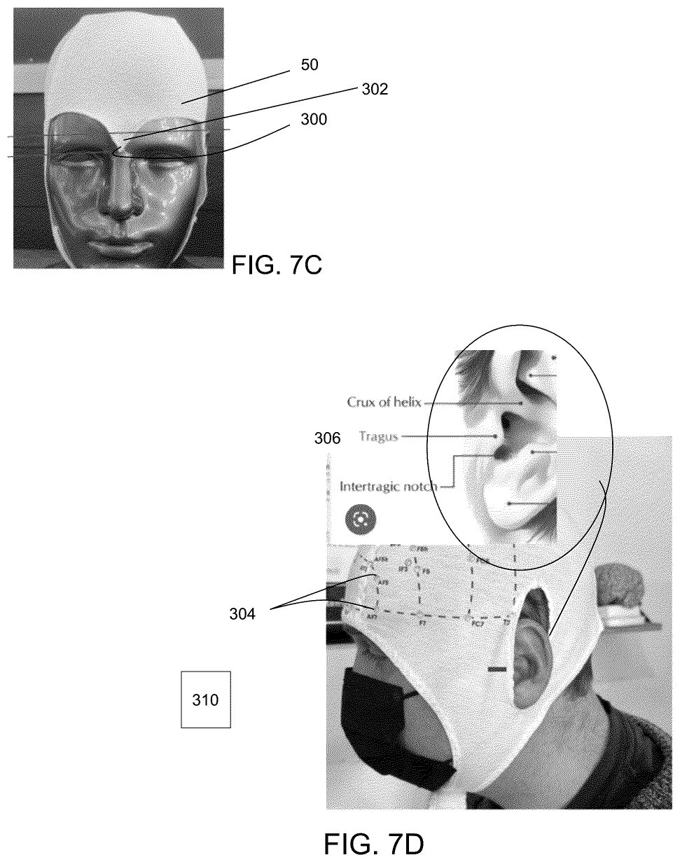

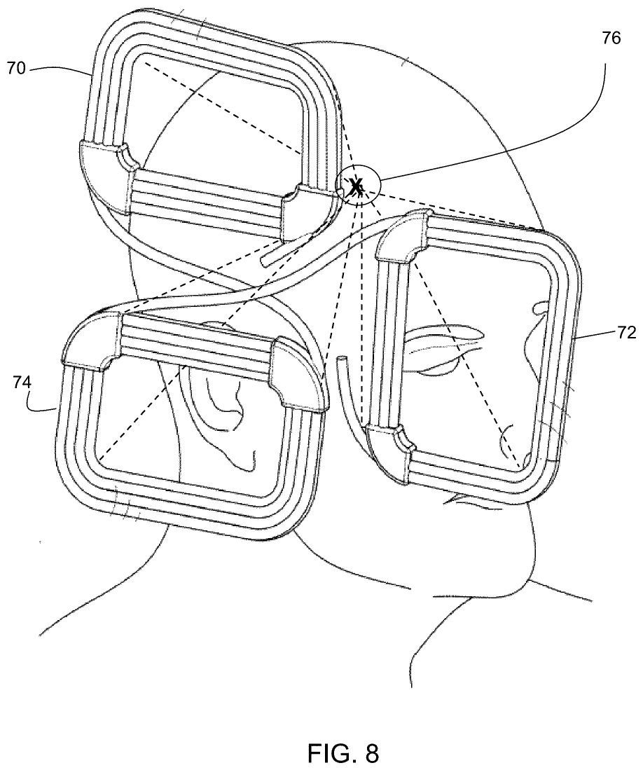

Referring also to FIGS. 6A and 6B, in one embodiment of the disclosure, we provide a treatment cap 50 sized and shaped to fit snugly over a patient's head. The cap may be composed of material intentionally designed to stretch, to accommodate a defined range of head sizes slightly larger than its unstretched size. Preferably cap 50 may be provided in a kit with several different sizes to fit different size patients. Typically, five sizes are sufficient to fit the majority of adult heads, a sixth size for youths, and a seventh size for small children and infants. Cap 50 includes indicia 52 in the form of a specific grid with anatomical markers printed on the cap. The indicia or markers may include text, color and/or symbols to identify specific target locations in the head of the wearer and/or shapes and patterns to indicate orientation for the TMS windings 12 to facilitate the magnetic induction in the correct location and orientation. These indicia may also be comprised of symbols, QR codes, color spectra, or any combination thereof. The indicia may be identical across numerous copies of the cap produced. Alternatively, cap 50 may instead have unique indicia 57 in one or more locations such that the cap and any location on it can be uniquely distinguished from any other (FIG. 7A). Alternatively, cap 58A may have printed patterns or color gradations to guide placement (FIG. 7B). The caps also may include indicia to personalize a cap to an individual patient.

As mentioned supra, the current standard of care is to place the treatment cap just above the eyebrows, measure the distance from nasion to cap brim, and then for every subsequent session, try and place the cap back exactly that same distance, remeasuring each time. This is laborious and error prone.

Referring to FIGS. 7C and 7D, in accordance with another embodiment the present disclosure rather than use a cap with a flat brim 300, the present disclosure uses unique cap geometry where the brim 300 comes to a point 302 on the midline. This point can be immediately visually confirmed to be correctly placed or not without the need for measuring tape.

Additionally, we may place indicia 304 on the cap that denotes where the cap should be with respect to the tragus 306 (the point flap of skin on the ear), as an additional marker to ensure reliable fit of the cap on the head.

With both the pointed brim 300, and tragus markers 306 on both sides, these three markers may be used not only for basic visual confirmation, but an AI algorithm optionally can be implemented to ensure proper cap positioning, using a smartphone 310 camera and slowly wave it around the patient from left to front to right side to ensure the cap is properly positioned.

Because the magnetic induction coil's field has a particular orientation (it is directional, not symmetric), the angle at which the magnetic induction coil is placed over a given location makes a meaningful difference in where the focal point of the coil(s) land and in turn the success of the treatment and how patients experience the procedure. Specifically, even over the exact same central location, positioning the coil at different angles will activate different central and peripheral nerves. In the latter case, this may cause uncomfortable sensations at some angles, but not others. For example, at some angles, a patient's jaw may jitter during TMS, while not at others. Thus, the indicia's shape and pattern uniquely identify each angle at which the magnetic induction coil may be placed so that, in conjunction with the camera, a viewer can see if they are properly and consistently aligned. Notably, the indicia are neither radially, nor bilaterally symmetric, and thus a rotation of 180 degrees of the magnetic induction coil will result in a different perspective on any given marker so that it is again, uniquely identified. Similarly, the text and color combination of each anatomical marking uniquely identifies the location. Locations commonly used as stimulation targets or reference locations in the therapeutic TMS community are further differentiated using color, to allow for quick and robust setup. This permits the healthcare provider to ensure that the magnetic induction windings 12A, 12B are properly positioned on the head of the wearer, and not skewed or tilted. We also can infer the coil's distance from the head of the wearer due to image size, to ensure that the coil is in full contact when seen by the imaging device.

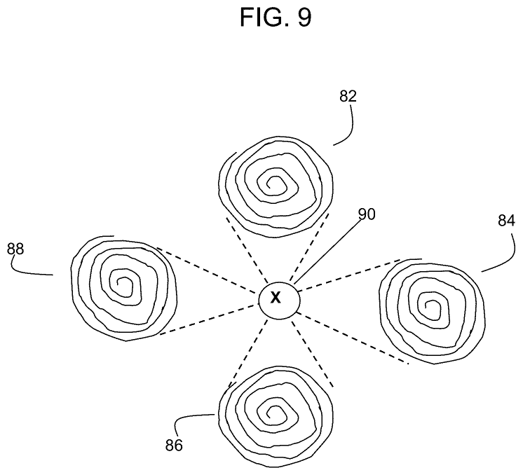

While the foregoing disclosure illustrates the advantage of employing a single imaging device between two similar coils in a two coil TMS head system, by the same token, providing an imaging device, i.e., a camera directly over the focal point of TMS coils head as above described advantageously may be employed with other coil head designs. For example, as shown in FIG. 8, a TMS coil head in accordance with the teaching of US 2016/0206896 A1 may comprise three coils 70, 72, 74 having an imaging device camera 76 located directly over the focal point of the three coils.

Referring to FIG. 9, in another embodiment, a TMS coil head may comprise four coils 82, 84, 86, 88 having a camera 90 directly over the focal point of the four coils.

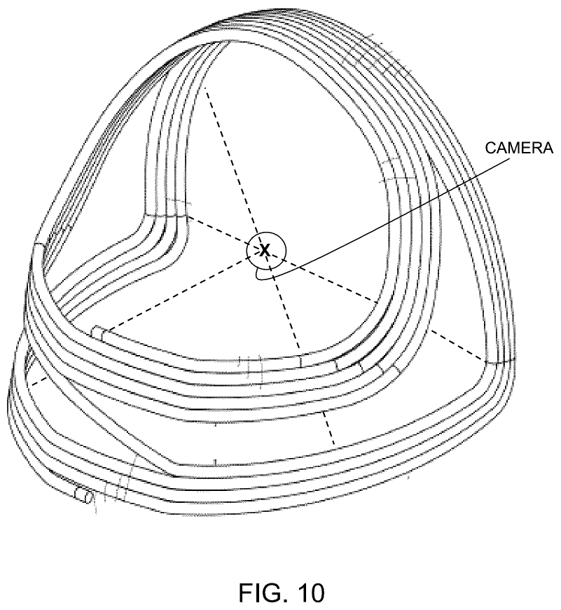

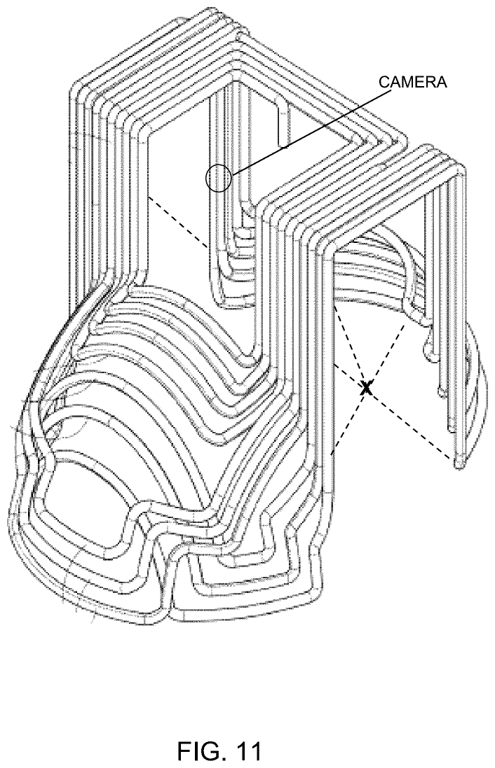

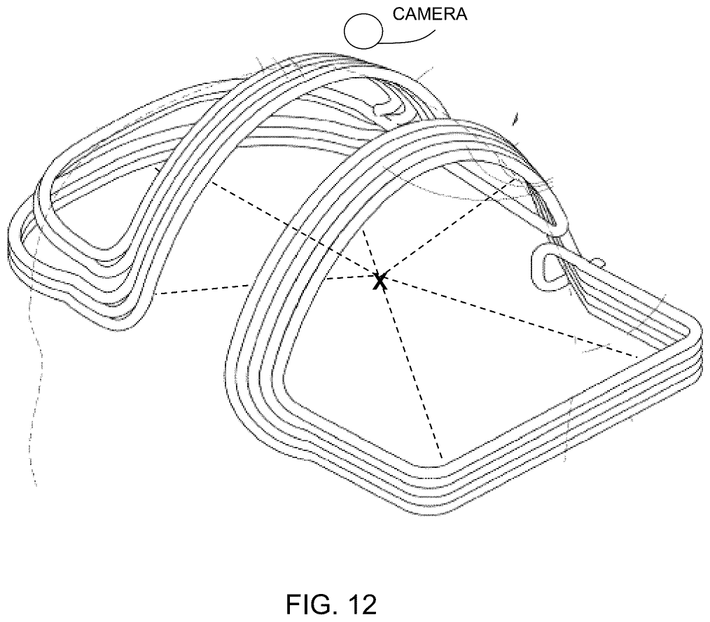

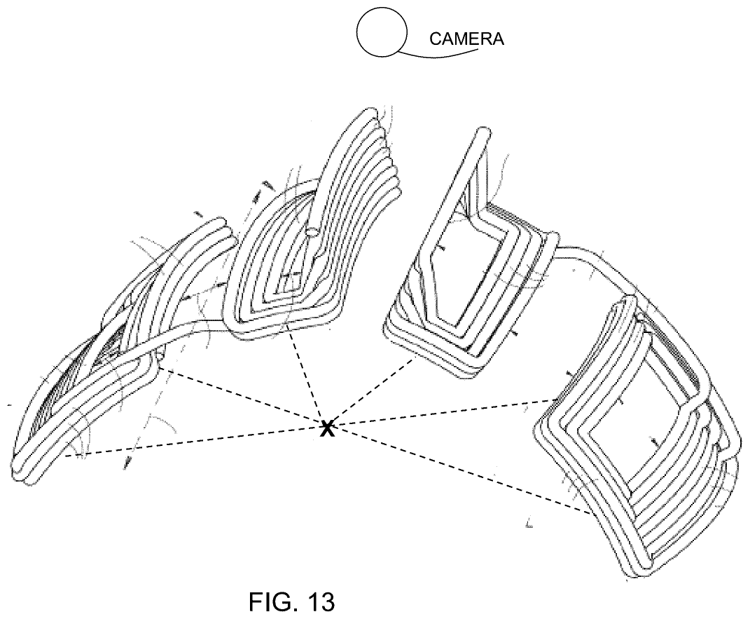

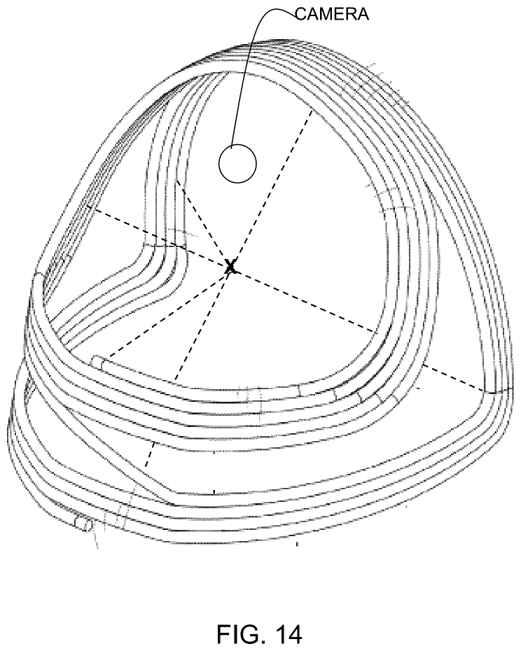

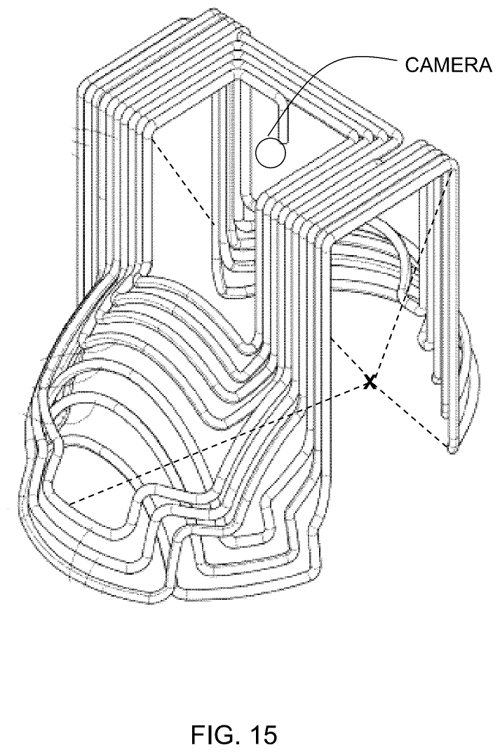

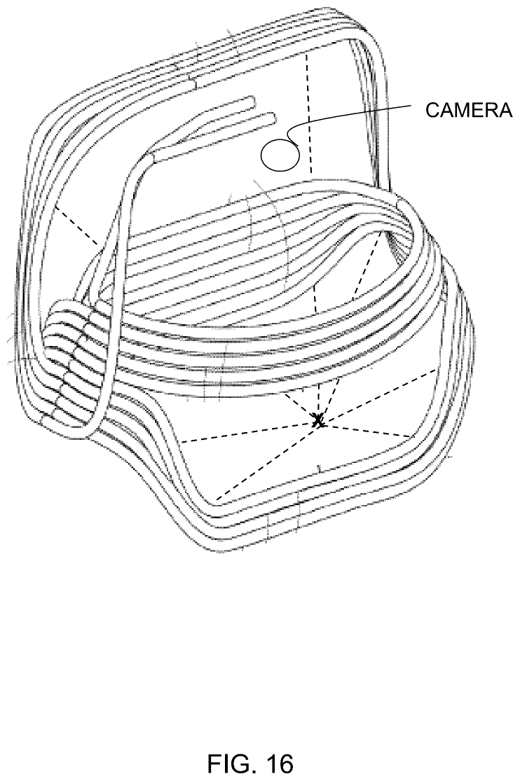

FIGS. 10-16 illustrate still other more complicated coil head geometrics taken from US Published Application Nos. US 2016/0206895 A1 (FIGS. 7 and 8), US 2016/0206896 A1 (FIGS. 11 and 13B), US 2014/0235926 A1 (FIGS. 7 and 8) and US 2014/0235927 A1 (FIG. 8), respectively, the contents of which are incorporated herein by reference, which may benefit by having a single camera directly over the focal point of the coil(s) in accordance with the subject disclosure.

Various changes may be made in the foregoing disclosure without departing from the spirit and scope thereof.

What is claimed:

1. A transcranial magnetic stimulation (TMS) coil head configured to be placed over a target brain region for treatment, wherein the TMS coil head comprises a housing containing one or more coil windings within the housing, wherein said one or more coil windings are configured to generate a maximum magnetic field at a common focal point; and one or more imaging devices including a single camera configured to overlie the common focal point is as to permit direct visualization of a position of the TMS coil head relative to a target position on a patient's head.

2. The TMS coil head of claim 1, wherein the TMS coil head comprises two coil windings located to either side of a center of the TMS coil head.

3. The TMS coil head as claimed in claim 2, wherein the two coil windings are mirror images of one another.

4. The TMS coil head of claim 1, wherein one or more imaging devices comprises one or more cameras, including the single camera overlying the common focal point of the one or more coil windings.

5. The TMS coil head of claim 1, wherein the one or more imaging devices comprises one or more visible light imaging cameras, one or more ultraviolet light imaging cameras or one or more infrared imaging cameras.

6. The TMS coil head of claim 1, wherein the one or more imaging devices also comprises two or more cameras located to sides of the TMS coil head.

7. The TMS coil head of claim 1, wherein the one or more imaging devices also comprises two or more cameras located away from the center but within the housing of the TMS coil head.

8. The TMS coil head of claim 1, further comprising one or more accelerometers configured to sense orientation placement or changes in orientation of the TMS coil head.

9. The TMS coil head of claim 1, further comprising one or more contact sensors configured to detect contact and force between the TMS coil head and the patient's head.

10. The TMS coil head of claim 9, wherein the contact sensors comprise one or more force-sensitive resistors, one or more capacitive touch sensors or one or more ultrasonic position/touch sensors.

11. The TMS coil head of claim 1, further comprising one or more imaging devices configured to permit simultaneous visualization of the patient's head as well as the TMS coil head.

12. The TMS coil head of claim 11, wherein the one or more imaging devices comprises one or more cameras, one or more LIDAR detectors, or one or more ultrasonic detectors.

13. The TMS coil head of claim 1, further comprising a memory device configured to create a record of the TMS coil head position before and during treatment.

14. A transcranial magnetic stimulation (TMS) kit comprising a transcranial magnetic stimulation system, comprising:
i) a pulse generator;
ii) a TMS coil head as claimed in claim 1 configured to be placed over a target brain region of a patient for treatment; and
(iii) a patient head cap having at least one of grid markings, text and color markings configured to overlie anatomical locations on the head of the patient.

15. The TMS kit of claim 14, including markings configured to overlie target areas of the head of the patient.

16. The TMS kit of claim 14, including markings configured to permit continuous measurement of a relative position and orientation of the cap versus the patient's head before, during, and after treatment.
